(19) United States
(12) Patent Application Publication
(10) Pub. No.: US 2024/0018020 A1
(43) Pub. Date: Jan. 18, 2024

(54) PURIFICATION METHOD AND PURIFICATION APPARATUS FOR LIQUID TO BE PROCESSED CONTAINING TETRAALKYLAMMONIUM IONS

(71) Applicant: ORGANO CORPORATION, Tokyo (JP)

(72) Inventors: Noriko TAKADA, Tokyo (JP); Kaoru NUKUI, Tokyo (JP); Haruo YOKOTA, Tokyo (JP)

(73) Assignee: ORGANO CORPORATION, Tokyo (JP)

(21) Appl. No.: 18/036,043

(22) PCT Filed: Sep. 27, 2021
(86) PCT No.: PCT/JP2021/035325

§ 371 Date: May 9, 2023

(30) Foreign Application Priority Data
Nov. 10, 2020 (JP) 2020-187291

(51) Int. Cl. C02F 1/42; B01J 39/05; B01J 49/53; C07C 211/63

(52) U.S. Cl. CPC C02F 1/42; B01J 39/05; B01J 49/53; C07C 211/63; C02F 2101/38

(57) ABSTRACT

The present invention provides a method for purifying a liquid to be treated, the method being capable of reducing the content of metal impurities in the liquid to be treated containing tetraalkylammonium ions, and further, being capable of suppressing cracking of the resin even when a strongly acidic cation exchange resin is used. The method includes: an impurity removal step of passing a liquid to be treated that contains tetraalkylammonium ions and metal impurities through a container filled with a cation exchange resin of a hydrogen ion form or a tetraalkylammonium ion form to reduce the content of the metal impurities in the liquid to be treated, wherein the degree of cross-linking of the cation exchange resin is from 16% to 24%.

### TECHNICAL FIELD

The present invention relates to a method and an apparatus for purifying a liquid to be treated, which reduce the content of metal impurities in the liquid to be treated containing tetraalkylammonium ions and metal impurities. The present invention also relates to a method and an apparatus for recovering an aqueous solution of a tetraalkylammonium salt from a liquid to be treated, which reduce the content of metal impurities in the liquid to be treated containing tetraalkylammonium ions and metal impurities.

### BACKGROUND ART

In the manufacture of electronic components such as semiconductor devices, liquid crystal displays, and printed circuit boards, a photoresist film is formed on a substrate such as a wafer, irradiated with light or the like through a pattern mask, and then the unnecessary photoresist is dissolved and developed by a developer. After further processing such as etching, the insoluble photoresist film on the substrate is peeled off. The photoresist has a positive type in which the exposed portion becomes soluble and a negative type in which the exposed portion becomes insoluble, and an alkaline developer is mainly used as a developer of the positive photoresist. As the developer of the negative photoresist, an organic solvent-based developer is mainly used, but an alkaline developer may be used in some cases.

An aqueous solution of tetraalkylammonium hydroxide (hereinafter also referred to as “TAAH”) is usually used as the alkaline developer. Therefore, the waste liquid discharged in the developing step of the photoresist (hereinafter, also referred to as “photoresist developing waste liquid”) contains metal ions (metal impurities) and tetraalkylammonium ions (hereinafter, also referred to as “TAA ions”) in addition to the photoresist.

Conventionally, the main methods for treating a photoresist developing waste liquid have been to concentrate them by an evaporation method or a reverse osmosis membrane method and dispose (incineration or pickup by a vendor) of them, or biodegrade by an activated sludge and discharge them. However, from the viewpoint of reducing the environmental burden, an attempt to recover and reuse TAAH from the photoresist developing waste liquid has also been proposed.

Patent Document 1 discloses a method for recovering TAA ions by adsorbing them on a cation exchange resin and then eluting the TAA ions as tetraalkylammonium salts (hereinafter, also referred to as “TAA salts”) using an acid solution. In Patent Document 1, in the process of recovering the TAA salt solution, the pH and/or the electric conductivity of the effluent are measured, and the recovery is stopped when they change by a predetermined amount to obtain a TAA salt solution having a reduced metal ion concentration. Then, TAAH is produced using the TAA salt solution as a raw material.

### PRIOR-ART DOCUMENT

### Patent Document

[Patent Document 1] WO 2012/090699 A1

### SUMMARY OF THE INVENTION

### Technical Problems

However, in the method described in Patent Document 1, the recovered TAA salt solution is finally evaporated and concentrated, and then electrolyzed to obtain TAAH, and in the concentration step, the metal ions remaining in the TAA salt solution cause scaling.

On the other hand, as a method for reducing the amount of metal impurities, generally, a method of adsorbing the metal impurities with a strongly acidic cation exchange resin is effectively used. However, the strongly acidic cation exchange resin in the tetraalkylammonium ion form swells due to more water content in the resin than in the hydrogen ion form. Therefore, repeated conversion between the hydrogen ion form and the tetraalkylammonium ion form causes cracks due to repeated shrinkage and swelling, resulting in resin rupture.

Therefore, an object of the present invention is to provide a method and an apparatus for purifying a liquid to be treated, the method and the apparatus being capable of reducing the content of metal impurities in the liquid to be treated containing tetraalkylammonium ions, and further, being capable of suppressing cracking of the resin even when a strongly acidic cation exchange resin is used. Another object of the present invention is to provide a method and an apparatus for recovering an aqueous solution of a tetraalkylammonium salt from a liquid to be treated containing tetraalkylammonium ions, the method and the apparatus being capable of suppressing cracking of the resin even when a strongly acidic cation exchange resin is used.

### Means for Solving the Problems

In view of the above problems, as a result of intensive studies by the present inventors, it was found that the cracking of the resin can be suppressed and that the content of the metal impurities in the liquid to be treated containing tetraalkylammonium ions can be reduced by using a strongly acidic cation exchange resin having a high degree of cross-linking, and the present invention has been completed.

That is, the present invention is a method for purifying a liquid to be treated, the method including:
an impurity removal step of passing a liquid to be treated containing tetraalkylammonium ions and metal impurities through a container filled with a cation exchange resin of a hydrogen ion form or a tetraalkylammonium ion form to reduce a content of the metal impurities in the liquid to be treated,
wherein a degree of cross-linking of the cation exchange resin is from 16% to 24%.

Further, the present invention is an apparatus for purifying a liquid to be treated, the apparatus including:
an impurity removal means for passing a liquid to be treated containing tetraalkylammonium ions and metal impurities through a container filled with a cation exchange resin of a hydrogen ion form or a tetraalkylammonium ion form to reduce a content of the metal impurities in the liquid to be treated,
wherein a degree of cross-linking of the cation exchange resin is from 16% to 24%.

Further, the present invention is a method for recovering an aqueous solution of a tetraalkylammonium salt from a liquid to be treated, the method including:
an impurity removal step of passing a liquid to be treated containing tetraalkylammonium ions and metal impurities through a container filled with a cation exchange resin of a hydrogen ion form or a tetraalkylammonium ion form to reduce a content of the metal impurities in the liquid to be treated,
wherein a degree of cross-linking of the cation exchange resin is from 16% to 24%.

Furthermore, the present invention is an apparatus for recovering an aqueous solution of a tetraalkylammonium salt from a liquid to be treated, the apparatus including:
an impurity removal means for passing a liquid to be treated containing tetraalkylammonium ions and metal impurities through a container filled with a cation exchange resin of a hydrogen ion form or a tetraalkylammonium ion form to reduce a content of the metal impurities in the liquid to be treated,
wherein a degree of cross-linking of the cation exchange resin is from 16% to 24%.

### Effect of the Invention

According to the present invention, it is possible to provide a method and an apparatus for purifying a liquid to be treated that reduce the content of metal impurities in the liquid to be treated containing tetraalkylammonium ions, in which cracking of the resin can be suppressed by using a highly cross-linked strongly acidic cation exchange resin. According to the present invention, it is possible to provide a method and an apparatus for recovering an aqueous solution of a tetraalkylammonium salt from a liquid to be treated containing tetraalkylammonium ions, in which cracking of the resin can be suppressed by using a highly cross-linked strongly acidic cation exchange resin. In addition to the above, when a highly cross-linked strongly acidic cation exchange resin with a small particle diameter is used, it is possible to provide a method and an apparatus for purifying a liquid to be treated and a method and an apparatus for recovering an aqueous solution of a tetraalkylammonium salt from the liquid to be treated, with less pH variation at the early stage of the liquid flow.

### BRIEF DESCRIPTION OF THE DRAWINGS

FIG. 1 is a schematic diagram showing a configuration of a purification apparatus according to one embodiment of the present invention.

FIG. 2 is a schematic diagram showing a configuration of a purification apparatus according to one embodiment of the present invention.

### DETAILED DESCRIPTION OF THE PRESENT INVENTION

### <Method for Purifying Liquid to be Treated>

The purification method according to the present invention includes an impurity removal step of passing a liquid to be treated containing tetraalkylammonium ions and metal impurities through a container filled with a cation exchange resin of a hydrogen ion form (hereinafter, also referred to as “H-form”) or a tetraalkylammonium ion form (hereinafter, also referred to as “TAA-form”) to reduce the content of the metal impurities in the liquid to be treated. Furthermore, the purification method according to the present invention is characterized in that the degree of cross-linking of the cation exchange resin is from 16% to 24%. Hereinafter, the purification method according to the present invention will be described in detail.

### Impurity Removal Step

The impurity removal step is a step of passing a liquid to be treated containing tetraalkylammonium ions and metal impurities through a container filled with an H-form or a TAA-form cation exchange resin to reduce the content of the metal impurities in the liquid to be treated.

### (Liquid to be Treated)

In the present invention, the liquid to be treated containing tetraalkylammonium ions and metal impurities is not particularly limited as long as it contains at least tetraalkylammonium ions and metal impurities. However, since these components are contained and are generated in a large amount in a semiconductor manufacturing process, a liquid crystal display manufacturing process, or the like, it is preferable that the liquid to be treated is a solution derived from the photoresist developing waste liquid discharged in the process. The photoresist developing waste liquid is a waste liquid discharged when the photoresist after exposure is developed with an alkaline developer and is usually an aqueous solution having an alkaline pH of from 10 to 14. Therefore, in the photoresist developing waste liquid, the photoresist dissolves in the form of salts with TAA ions derived from TAAH by dissociating its acidic groups such as carboxyl groups and phenolic hydroxyl groups. Thus, photoresist developing waste liquid is a solution that mainly contains photoresist, TAA ions, and metal impurities. The liquid to be treated according to the present invention is, for example, a solution in which TAA ions in the photoresist developing waste liquid are adsorbed on a cation exchange resin and then recovered as TAA salts by eluting the TAA ions with an acid such as hydrochloric acid.

That is, first, the photoresist developing waste liquid is passed through the container filled with an H-form cation exchange resin, and TAA ions are adsorbed on the cation exchange resin. Here, since the usual metal ions contained in the waste liquid are also cations, the metal ions are adsorbed on the cation exchange resin through this passage. In the case where the ionic species containing the metal ion itself becomes an anion due to a chemical equilibrium reaction such as complexation in the waste liquid, the metal ions are not adsorbed on the cation exchange resin and are discharged from the container. On the other hand, since organic components derived from the photoresist dissolved in the resist developing waste liquid are usually in the form of anions, which are not easily adsorbed on the cation exchange resin, so most of them can be removed. In addition, if non-ionic components are present, most of them can be removed because they are not adsorbed on the cation exchange resin at this stage and are discharged (flow out). After the photoresist developing waste liquid is passed through the cation exchange resin, the photoresist components and other impurities remaining slightly in the resin may be washed by flowing ultrapure water or a highly pure TAAH aqueous solution.

Thereafter, a mineral acid aqueous solution such as hydrochloric acid or sulfuric acid is passed through the container filled with the cation exchange resin converted into the TAA-form, whereby the hydrogen ions contained in the mineral acid aqueous solution are sequentially replaced with the adsorbed TAA ions, and TAA ions flow out of the container as the acid salt (TAA salt) of the mineral acid used. In addition, the solution containing the obtained TAA salt can be treated with a (highly cross-linked) cation exchange resin (preferably having a small particle diameter) to obtain a liquid to be treated according to the present invention. The liquid to be treated thus obtained is a solution containing tetraalkylammonium ions and metal impurities, and the purification method according to the present invention is a purification method for reducing the content of metal impurities in the liquid to be treated.

Note that the step of recovering TAAH in the photoresist developing waste liquid as a liquid to be treated containing TAA salts is known, for example, as described in Patent Document 1, and a known method can be appropriately selected and used for a container, a cation exchange resin, a type or an amount of an acid, a method of passing an acid, and the like used in the step. Here, as the (highly cross-linked) cation exchange resin used in the step, it is also possible to use a strongly acidic cation exchange resin having the degree of cross-linking of from 16% to 24% according to the present invention. In this case, even in this step, it is possible to prevent cracking of the resin due to repeated use. Further, the same resin can be used from the step of recovering the liquid to be treated to the ion exchange step and the impurity removal step described later, and is also preferable from the viewpoint of operability.

### (Tetraalkylammonium Ion)

As described above, the liquid to be treated used in the present invention is a solution obtained by eluting TAA ions (TAAH) as TAA salts from the photoresist developing waste liquid and recovering the eluted solution. Specific examples of TAA ions in the liquid to be treated include ions derived from tetraalkylammonium hydroxides such as tetramethylammonium hydroxide, tetraethylammonium hydroxide, tetrapropylammonium hydroxide, tetrabutylammonium hydroxide, methyltriethylammonium hydroxide, trimethylethylammonium hydroxide, dimethyldiethylammonium hydroxide, trimethyl(2-hydroxyethyl)ammonium hydroxide, triethyl(2-hydroxyethyl)ammonium hydroxide, dimethyldi(2-hydroxyethyl)ammonium hydroxide, diethyldi(2-hydroxyethyl)ammonium hydroxide, methyltri(2-hydroxyethyl)ammonium hydroxide, ethyltri(2-hydroxyethyl)ammonium hydroxide, and tetra(2-hydroxyethyl)ammonium hydroxide, as alkalis used in the photoresist developers. Among these, tetramethylammonium ions and tetrabutylammonium ions derived from tetramethylammonium hydroxide and tetrabutylammonium hydroxide, which are most commonly used, are preferably used in the present invention, and tetramethylammonium ions derived from tetramethylammonium hydroxide are particularly preferably used. The liquid to be treated used in the present invention is a solution obtained by recovering the tetraalkylammonium ions as, for example, chloride salts, and is preferably an aqueous solution of tetraalkylammonium chloride such as tetramethylammonium chloride or tetrabutylammonium chloride and is more preferably an aqueous solution of tetramethylammonium chloride. That is, the tetraalkylammonium ions contained in the liquid to be treated according to the present invention are preferably tetraalkylammonium ions derived from tetraalkylammonium chlorides such as tetramethylammonium chloride and tetrabutylammonium chloride and are more preferably tetraalkylammonium ions derived from tetramethylammonium chloride.

Here, a typical photoresist developing waste liquid discharged from a developing process in semiconductor manufacturing and liquid crystal display manufacturing will be described. In the developing process, a single-wafer type automatic developing device is usually used extensively. In this device, a step of using a developer containing TAAH and a step of subsequent rinsing (substrate cleaning) with pure water are performed in the same tank, and in the rinsing step, pure water in an amount of 5 to 1000 times that of the developer is used. Therefore, the developer used in the developing process is usually a waste liquid diluted 5 to 10 times. Consequently, the composition of the photoresist developing waste liquid discharged in the developing process is about 0.001 mass % to 2.5 mass % of TAAH, about 10 to 100 ppm of the resist, and about 0 to several 10 ppm of the surfactant. In addition, the waste liquid of another process may be mixed in, and TAAH concentration may be lowered even within the above-described range. The TAA ion concentration of the liquid to be treated obtained from the photoresist developing waste liquid having a TAAH concentration of, for example, from 0.001 mass % to 2.5 mass % is from 0.001 mass % to 2.5 mass %. Note that the liquid to be treated obtained from the photoresist developing waste liquid may be used after adjusting TAA ion concentration by appropriately performing concentration or the like.

### (Metal Impurities)

Since the photoresist developing waste liquid contains a plurality of metal ions as metal impurities, the liquid to be treated also contains the metal ions. Examples of the metal ion include monovalent ions such as sodium and potassium; divalent ions such as magnesium, calcium, and zinc; and polyvalent ions such as aluminum, nickel, copper, chromium, and iron. These metal ions are usually contained in the photoresist developing waste liquid (liquid to be treated) in the range of from 0.1 ppb to 1000 ppb. Note that the counter ions of the tetraalkylammonium ions in the photoresist developing waste liquid are usually hydroxide ions, but in some factories, or when neutralization is performed, at least one selected from inorganic anions such as fluoride ion, chloride ion, bromide ion, carbonate ion, hydrogen carbonate ion, sulfate ion, hydrogen sulfate ion, nitrate ion, phosphate ion, hydrogen phosphate ion, and dihydrogen phosphate ion and organic anions such as formate ion, acetate ion, oxalate ion is generally at least a part of the counter ions of the tetraalkylammonium ions. However, since most of these anions are removed at the stage of preparing the liquid to be treated from the photoresist developing waste liquid, it is considered that these anions are hardly contained in the liquid to be treated.

### (Cation Exchange Resin)

In the present invention, the strongly acidic cation exchange resin having a degree of cross-linking of from 16% to 24% is used as the cation exchange resin of the H-form or TAA-form. The highly cross-linked resin with the degree of cross-linking in the above range has a high strength due to the presence of a dense cross-linked structure inside the resin. When a cation exchange resin having the degree of cross-linking of less than 16% is used, the strength of the resin becomes insufficient and there is a high possibility that cracking of the resin occurs during purification. In addition, when a cation exchange resin having the degree of cross-linking of more than 24% is used, the ion exchange rate becomes slow or the regeneration rate of the resin becomes slow. As described above, in the present invention, it has been found that cracking of the resin during purification can be suppressed by using the strongly acidic cation exchange resin having the degree of cross-linking of from 16% to 24%. The highly cross-linked cation exchange resin is also preferable in that they have a high exchange capacity and can introduce more functional groups.

As the H-form cation exchange resin, any resin can be used as long as the degree of cross-linking is from 16% to 24%. Examples of such H-form cation exchange resins include Amberjet (registered trademark) 1060H and 1600H (trade name, manufactured by Organo Corporation), AMBERLITE (registered trademark) IRN99H, 200C, and 200CT (trade name, manufactured by Du Pont de Nemours, Inc.), AMBEREX 210 (trade name, manufactured by Du Pont de Nemours, Inc.), Diaion (registered trademark) SK116 (trade name, manufactured by Mitsubishi Chemical Corporation), Purolite (registered trademark) C100X16MBH (trade name, manufactured by Purolite K. K.), and the like.

As the TAA-form cation exchange resin, the resin that have been ion-exchanged in advance to the TAA-form from the resins exemplified as H-form cation exchange resins above. That is, in the impurity removal step, when the liquid to be treated is purified using the cation exchange resin of TAA-form, the purification method according to the present invention may include the following ion exchange step prior to the impurity removal step.

An ion exchange step of passing a regeneration agent containing tetraalkylammonium ions through a container filled with a cation exchange resin of a hydrogen ion form to convert the cation exchange resin of the hydrogen ion form into a cation exchange resin of the tetraalkylammonium ion form.

Then, the cation exchange resin of TAA-form obtained in the ion exchange step can be used in the impurity removal step. The ion exchange step will be described later.

The particle diameter of the cation exchange resin is preferably from 200 μm to 720 μm. The particle diameter of 720 μm or less is within the particle diameter range of common ion exchange resins, making it easy to convert and operate existing facilities. In addition, the cation exchange resin having a particle diameter of 200 μm or more has a general surface area and can sufficiently remove metal impurities. In addition, when the cation exchange resin has a particle diameter of 200 μm or more, an increase in the differential pressure between the resin outlet and the resin inlet can be suppressed. Furthermore, the particle diameter of the cation exchange resin is more preferably from 500 μm to 560 μm in the H-form. The small particle diameter cation exchange resin with the particle diameter in this range is easily converted from an H-form to a TAA-form due to the large surface area of the resin. Therefore, the amount of the H-form resin remaining when the resin is converted into the TAA-form is reduced, and pH variation at the early stage when passing through the liquid to be treated can be further suppressed. In addition, the cation exchange resin having a small particle diameter has a large surface area of the resin, and thus is excellent in removal performance of the metal impurities. Note that the particle diameter means a harmonic average diameter in the present invention.

### (When Using Cation Exchange Resin of H-Form)

When the liquid to be treated containing TAA ions and metal impurities is passed through a container filled with the cation exchange resin of the H-form, hydrogen ions in the resin and TAA ions in the liquid to be treated are ion-exchanged, whereby the H-form cation exchange resin is converted into the TAA-form cation exchange resin. Further, since the metal impurities which are cations in the liquid to be treated are also adsorbed on the cation exchange resin, the content of the metal impurities in the liquid to be treated can be reduced. In other words, when the cation exchange resin of the H-form is used, the cation exchange resin can be converted from H-form to TAA-form using the liquid to be treated, which is to be purified, without separately performing an ion exchange step described later. In this way, the cation exchange resin converted into the TAA-form can subsequently be used in the impurity removal step. As the ion form of the cation exchange resin after the implementation of this step, the TAA-form and the metal ion form are mixed. In the case where an unreacted exchange group remains, a cation exchange resin of the hydrogen ion form is also mixed.

When using the cation exchange resin of the H-form, it is possible to reduce the content of the metal impurities in the liquid to be treated by passing the liquid to be treated through the resin once. In order to improve the purification efficiency, the cation exchange resin that has been converted into the TAA-form (and the metal ion form) by the first pass of the liquid to be treated may be passed through the liquid to be treated again. That is, the impurity removal step may be repeated a plurality of times. When the liquid to be treated is passed through the cation exchange resin that has been converted into the TAA-form (and the metal ion form) again, TAA ions adsorbed on the resin and the metal ions remaining in the liquid to be treated are ion-exchanged, and the metal ions are adsorbed on the resin, whereby the content of the metal impurities in the liquid to be treated can be further reduced.

In addition, when H-form cation exchange resin is used to pass through the liquid to be treated, the pH of the effluent flowing out of the container becomes strongly acidic due to the effect of the hydrogen ions flowing out of the cation exchange resin. Therefore, in this case, the purification method according to the present invention may include a neutralization step of neutralizing the effluent obtained in the impurity removal step. When the impurity removal step is repeated multiple times, for example, after the first impurity removal step, a neutralizing step of the effluent liquid to be treated can be performed, and the liquid to be treated with adjusted pH after the neutralization step can be used for the second impurity removal step. The neutralization step can be performed using a known method. Specifically, for example, it can be carried out by storing the effluent in a container such as a storage tank and adjusting the pH using an alkali such as TAAH. It should be noted that only the liquid to be treated which has flowed out in the early stage of the liquid flow with severe pH variation may be stored in a separate container such as a storage tank to adjust pH, and then mixed with the remaining liquid to be treated which has flowed out afterwards. In addition, the liquid to be treated which has flowed out in the early stage of the liquid flow with severe pH variation may be discarded. Examples of the alkali used for neutralization include tetramethylammonium hydroxide and ammonium hydroxide.

### (When Using Cation Exchange Resin of TAA-Form)

When the liquid to be treated containing TAA ions and metal impurities is passed through a container filled with the cation exchange resin of the TAA-form, TAA ions in the resin and the metal ions in the liquid to be treated are ion-exchanged, and the metal ions are adsorbed on the resin. As a result, the content of the metal impurities in the liquid to be treated can be reduced. When unreacted exchange groups (hydrogen ions) remain in the cation exchange resin in the ion exchange step, the hydrogen ions are also exchanged with the metal ions in the liquid to be treated in the present step. By using a cation exchange resin which has been converted from the H-form to the TAA-form in advance, TAA ions adsorbed on the resin rather than the hydrogen ions and the metal ions in the liquid to be treated will be exchanged when passing through the liquid to be treated. Therefore, variations in TAA ion concentration of the liquid to be treated and pH variation at the early stage of the liquid flow can be suppressed. As described above, from the viewpoint of suppressing pH variation at the early stage of the liquid flow and from the viewpoint of removal efficiency of the metal impurities, it is preferable to use a cation exchange resin of the TAA-form in the impurity removal step.

### (Passing of the Liquid to be Treated)

As a method of passing the liquid to be treated through the container filled with the cation exchange resin, a conventionally known method can be appropriately employed depending on the type and shape of the cation exchange resin. Here, in the present invention, the term “container” means any container, including but not limited to a “column” such as an adsorption column, or a “tank,” which can be filled with an ion exchange resin, and which can purify the liquid to be treated (either through water or a batch). Specifically, for example, a method (column method) in which a column having an inflow hole at an upper portion thereof and having an outflow hole at a lower end portion thereof is filled with a cation exchange resin, and a liquid to be treated is continuously passed through by using a pump, or a method (batch method) in which a liquid to be treated is passed through a container filled with a cation exchange resin, and the liquid to be treated is brought into contact with the resin for an appropriate time to remove the supernatant liquid. In the case of adopting the column method, the size of the column may be appropriately determined in accordance with the performance of the cation exchange resin and the like. From the viewpoint of efficient purification, for example, it is preferable that the ratio (L/D) between the height (L) and the diameter (D) of the column is from 0.5 to 30, and the space velocity (SV) of the liquid to be treated is 1 (1/hour) or more and 150 (1/hour) or less.

### (Recovery of Effluent)

When the column method is used to pass the liquid, the effluent in which the content of the metal impurities is reduced flows out from one end of the container by passing through the liquid to be treated containing tetraalkylammonium ions and the metal impurities and thus the effluent is collected in a storage tank or the like. The obtained purified liquid to be treated is an aqueous solution of a tetraalkylammonium salt. The content of metal impurities can be measured using, for example, Agilent 8900 triple quadrupole ICP-MS (trade name, manufactured by Agilent Technologies).

### Ion Exchange Step

The ion exchange step is a step of converting an H-form cation exchange resin into a TAA-form cation exchange resin prior to the impurity removal step described above, that is, the ion exchange step is a step of preparing a TAA-form cation exchange resin used in the impurity removal step. The ion exchange step is performed by passing a regeneration agent including TAA ions through a container filled with a cation exchange resin of an H-form. The H-form cation exchange resin is as described above. When the H-form cation exchange resin is passed through a regeneration agent that contains TAA ions, the hydrogen ions of the cation exchange resin and TAA ions contained in the regeneration agent undergo ion-exchange and TAA ions are adsorbed on the cation exchange resin. Consequently, the H-form cation exchange resin is converted into the TAA-form cation exchange resin.

### (Regeneration Agent Containing Tetraalkylammonium Ions)

The regeneration agent containing TAA ions may be any aqueous solution containing TAA ions, and is not particularly limited. Examples of the regeneration agents containing TAA ions include aqueous solutions of tetramethylammonium hydroxide, tetraethylammonium hydroxide, tetrapropylammonium hydroxide, tetrabutylammonium hydroxide, methyltriethylammonium hydroxide, trimethylethylammonium hydroxide, dimethyldiethylammonium hydroxide, trimethyl(2-hydroxyethyl)ammonium hydroxide, triethyl(2-hydroxyethyl)ammonium hydroxide, dimethyldi(2-hydroxyethyl)ammonium hydroxide, diethyldi(2-hydroxyethyl)ammonium hydroxide, methyltri(2-hydroxyethyl)ammonium hydroxide, ethyltri(2-hydroxyethyl)ammonium hydroxide, and tetra(2-hydroxyethyl)ammonium hydroxide. Among these, the most commonly used aqueous solution of tetramethylammonium hydroxide and aqueous solution of tetrabutylammonium hydroxide are preferably used in the present invention, and an aqueous solution of tetramethylammonium hydroxide is particularly preferably used.

The content of TAA ions in the regeneration agent can be, for example, from mass % to 25% mass %.

### (Passing of Regeneration Agent)

As for a method of passing a regeneration agent containing TAA ions through a container filled with a cation exchange resin, a conventionally known method can be appropriately employed depending on the type and shape of the cation exchange resin. Specifically, for example, a method (column method) in which a column having an inflow hole at an upper portion thereof and having an outflow hole at a lower end portion thereof is filled with a cation exchange resin, and a solution containing tetraalkylammonium ions is continuously passed through the column by using a pump, or a method (batch method) in which a solution is passed through a container filled with a cation exchange resin, and the solution is brought into contact with the resin for an appropriate time to remove the supematant liquid. In the case of adopting the column method, the size of the column may be appropriately determined in accordance with the performance of the cation exchange resin and the like. In order to adsorb TAA ions efficiently, for example, in a solution having the TAA ion content of from 0.1 mass % to mass %, it is preferable that the ratio (L/D) between the height (L) and the diameter (D) of the column is from 0.5 to 30, and the space velocity (SV) of the solution is 1 (1/hour) or more and 150 (1/hour) or less.

The amount of the regeneration agent to be passed through can be appropriately set in consideration of the exchange capacity of the cation exchange resin filled in the container. It is to be noted that whether TAA ions flow out (breakthrough) without being adsorbed on the resin by passing through a solution containing cations in an amount equal to or larger than the exchange capacity of the cation exchange resin can be confirmed by analyzing TAA ion concentration in the liquid flowing out through the container by the ion chromatography method. More conveniently, the height occupied by the cation exchange resin in the container may be measured. When the counter ion of the cation exchange resin is changed from hydrogen ion to TAA ion, the volume of the cation exchange resin is swollen to about twice, depending on the type of the cation exchange resin. Therefore, the adsorption of TAA ions can be confirmed by measuring the volume of the cation exchange resin. In addition, when the pH of the regeneration agent to be passed is alkaline of 10 or more, the pH of the passed liquid becomes alkaline when TAA ions pass through the container without being adsorbed, and thus can be confirmed by a pH meter. In addition, when TAA ions are contained in the liquid flowing out through the container, the electric conductivity of the liquid is usually increased, and thus can be confirmed by an electric conductivity meter.

### (Recovery of Effluent)

When the column method is used to pass the liquid, hydrogen ions ion-exchanged with TAA ions flow out from one end of the container by passing through the regeneration agent containing tetraalkylammonium ions, so that anions corresponding to the used regeneration agent (salt) flow out as counter ions, and thus the effluent is recovered in a storage tank or the like.

### Regeneration Step of Cation Exchange Resin

The purification method according to the present invention may include a regeneration step of regenerating the cation exchange resin that has been in contact with the liquid to be treated in the impurity removal step. Regeneration of the resin can be performed by contacting the resin with an acid by a known method to remove impurities such as metal ions and to convert the resin from the TAA ion form to the H-form. The resulting H-form cation exchange resin can be reused in the impurity removal step. The acid used in the regeneration step is not particularly limited as long as hydrogen ions are generated in the form of an aqueous solution, and examples thereof include aqueous mineral acids such as hydrochloric acid and sulfuric acid. Among these, hydrochloric acid is preferred from the viewpoint of being industrially available inexpensively and from the viewpoint of easy concentration adjustment. The concentration and the amount of hydrochloric acid to be used are not particularly limited as long as the concentration and the amount are sufficient for conversion to the H-form and removal of impurities such as metal ions. Typically, the resin can be converted from the TAA ion form to the H-form by contacting the above cation exchange resin with 3 to 20 (L/L-resin) of 1 mass % to 10 mass % hydrochloric acid. In the regeneration step, in addition to the washing using the mineral acid, the washing using ultrapure water or pure water may be performed as appropriate.

### <Apparatus for Purifying Liquid to be Treated>

The purification apparatus according to the present invention has an impurity removal means for passing a liquid to be treated containing tetraalkylammonium ions and metal impurities through a container filled with a cation exchange resin of a hydrogen ion form or a tetraalkylammonium ion form to reduce the content of the metal impurities in the liquid to be treated. Further, the purification apparatus according to the present invention is characterized in that the degree of cross-linking of the cation exchange resin is from 16% to 24%. The details of the impurity removal means are the same as the explanation of the impurity removal step in the purification method according to the present invention described above.

When a TAA-form cation exchange resin is used, the purification apparatus according to the present invention may include the following ion exchange means.

An ion exchange means for passing a regeneration agent containing tetraalkylammonium ions through a container filled with a cation exchange resin of a hydrogen ion form to convert the cation exchange resin of the hydrogen ion form into a cation exchange resin of the tetraalkylammonium ion form.

Then, the cation exchange resin of the TAA-form obtained by the ion exchange means can be used as the cation exchange resin in the impurity removal means. The details of the ion exchange means are the same as the explanation of the ion exchange step in the purification method according to the present invention described above.

When an H-form cation exchange resin is used, the purification apparatus according to the present invention may include a neutralization means for neutralizing the effluent obtained by the impurity removal means. The details of the neutralization means are the same as the explanation of the neutralization step in the purification method according to the present invention described above.

Further, the purification apparatus according to the present invention may include a regeneration means for regenerating the cation exchange resin that has been in contact with the liquid to be treated in the impurity removal means. The details of the regeneration means are the same as the explanation of the regeneration step in the purification method according to the present invention described above.

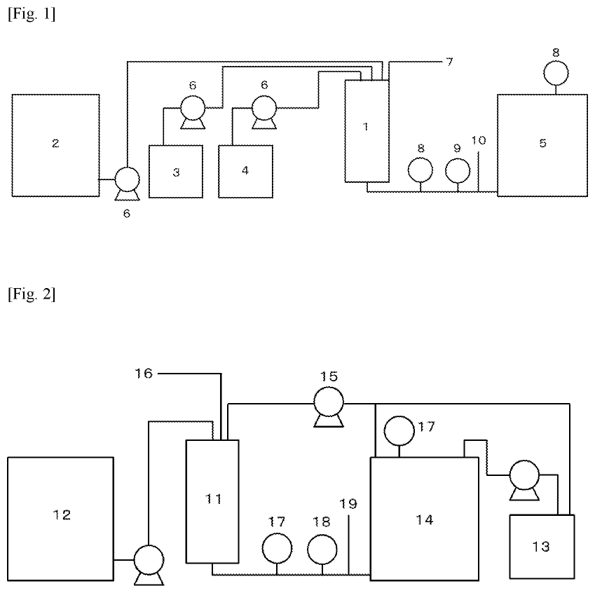

FIG. 1 is a schematic diagram showing an example of purification apparatus for purifying a liquid to be treated using a cation exchange resin adjusted to a TAA-form by an aqueous TAAH solution. Although FIG. 1 shows an example in which an adsorption column is used as a container filled with a cation exchange resin, the container is not limited to an adsorption column. First, as an ion exchange means, a regeneration agent including TAA ions (for example, an aqueous TAAH solution) is passed through the adsorption column 1 filled with the H-form cation exchange resin from the storage tank 3, and the effluent is recovered from the waste liquid line 10. Thereafter, as an impurity removal means, the liquid to be treated containing TAA ions and metal impurities is passed through the adsorption column 1 from the storage tank 2, and the effluent in which the content of metal impurities in the liquid to be treated is reduced is collected in the storage tank 5. Here, as shown in FIG. 1, the solution in the storage tank 2, the storage tank 3, and the storage tank 4 may be fed to the adsorption column 1 by the pump 6 for each solution, or may be fed to the adsorption column 1 by using one pump by switching with a valve.

The resin in the adsorption column 1 after use for purification can be reused by washing and regenerating as follows. Ultrapure water (or pure water) is passed through from ultrapure water (or pure water) line 7 to wash the resin in the adsorption column 1, and then an acid such as hydrochloric acid is passed through from the storage tank 4 to remove the metal impurities and TAA ions adsorbed on the resin, thereby making the resin H-form. The resin is then regenerated as the TAA-form cation exchange resin by passing a regeneration agent containing TAA ions (for example, an aqueous TAAH solution) from the storage tank 3 (corresponding to the ion exchange means). The regenerated TAA-form cation exchange resin can be reused as the TAA-form cation exchange resin used for the impurity removal means. Alternatively, ultrapure water (or pure water) can be passed through from the ultrapure water (or pure water) line 7 to wash the resin in the adsorption column 1 after being used for purification, and then reused as is as the TAA-form cation exchange resin used for the impurity removal means. However, in the latter case, where the resin is reused as is in the impurity removal means without passing hydrochloric acid, metal impurities which cannot be eluted by TAAH remain in the resin. Therefore, it is preferable to periodically combine the former regeneration method with hydrochloric acid flow. The waste liquid used for washing is discharged for each type of waste liquid according to the values of the pH meter 8 or the electric conductivity meter 9.

FIG. 2 is a schematic diagram showing an example of a purification apparatus for purifying a liquid to be treated using an H-form cation exchange resin. Although FIG. 2 shows an example in which an adsorption column is used as a container filled with a cation exchange resin, the container is not limited to an adsorption column. First, as an impurity removal means, a liquid to be treated, which contains TAA ions and metal impurities, is passed through the adsorption column 11 filled with the H-form cation exchange resin from the storage tank 12, and the effluent is collected in the storage tank 14. Since the obtained effluent becomes strongly acidic, the effluent may be neutralized as necessary. Specifically, an aqueous solution containing alkali (e.g., TAAH) is passed from the storage tank 13 to the storage tank 14 to neutralize the solution. Here, during the early stage of the flow of the liquid to be treated in the impurity removal step, the hydrogen ions in the H-form cation exchange resin are ion-exchanged with TAA ions or metal ions, and the pH of the effluent rapidly decreases. Therefore, if the strongly acidic solution flowing out in the early stage of the liquid flow is mixed in the storage tank 14 together with the effluent flowing out thereafter, the amount of alkali required for neutralization increases, which is not preferable. Therefore, it is preferable to confirm the pH of the effluent at the early stage of the liquid flow by a pH meter 17 installed in front of the waste liquid line 19 and discharge the strongly acidic effluent from the waste liquid line 19 in front of the storage tank 14. Further, in order to adjust pH of the liquid to be treated that has finally flowed out, the pH meter 17 is also installed in the storage tank 14. In the case where the impurity removal step is repeatedly performed, the liquid to be treated (neutralized as necessary) that has been flowed out is thereafter passed through the adsorption column 11 from the storage tank 14, and the effluent is again collected in the storage tank 14.

The resin in the adsorption column 11 after use for purification can be reused by washing and regenerating as follows. After ultrapure water (or pure water) is passed through from ultrapure water (or pure water) line 16 to wash the resin in adsorption column 11, the effluent collected in the storage tank 14 is passed through the adsorption column 11 to be regenerated as the TAA-form cation exchange resin. Alternatively, the resin in the adsorption column 11 is regenerated as the TAA-form cation exchange resin by passing ultrapure water (or pure water) from the ultrapure water (or pure water) line 16 to wash the resin, and then passing an aqueous TAAH solution from the storage tank 13. The TAA-form cation exchange resin regenerated in this way can be reused as the TAA-form cation exchange resin used for the impurity removal means. The former method can reduce the amount of chemicals used, but is inefficient in converting the resin to the TAA-form, considering the pH of the effluent. Therefore, in view of the efficiency of converting the resin into the TAA-form, the latter method is preferable. In addition, since metal impurities that cannot be completely eluted by TAAH remain in the resin, it is preferable to periodically combine the regeneration method with hydrochloric acid (not shown) flow as described with respect to the purification apparatus according to FIG. 1.

The purification apparatus according to the present invention can also be used in combination with an anion exchange resin or a particulate removal filter. When they are combined, the container filled with the anion exchange resin may be placed before and after the container filled with the cation exchange resin, or both ion exchange resins may be mixed and filled into the same container. Further, the container filled with the anion exchange resin is preferably installed in the preceding stage of the storage tank 5 or the storage tank 14. In addition, the particulate removal filter is preferably provided between the container filled with the cation exchange resin and/or the anion exchange resin and the storage tank 5 or the storage tank 14. As the anion exchange resin and the particulate removal filter, a known one can be appropriately selected and used, but the anion exchange resin is preferably converted into a Cl-form.

### <Method for Recovering Aqueous Solution of Tetraalkylammonium Salt>

The purification method according to the present invention is a method for purifying a liquid to be treated which reduces the content of metal impurities in the liquid to be treated containing tetraalkylammonium ions and metal impurities as described above, but the present invention can also be said to be a method for recovering a purified aqueous solution of a tetraalkylammonium salt from the liquid to be treated by reducing the content of metal impurities in the liquid to be treated containing tetraalkylammonium ions and metal impurities. That is, the liquid to be treated purified by the purification method according to the present invention is a recovered aqueous solution of a tetraalkylammonium salt. The aqueous solution of the tetraalkylammonium salt can then be contacted with, for example, an anion exchange resin or electrolyzed to obtain highly pure TAAH solution.

A method for recovering an aqueous solution of a tetraalkylammonium salt according to the present invention is a method for recovering an aqueous solution of a tetraalkylammonium salt from a liquid to be treated, the method including an impurity removal step of passing a liquid to be treated containing tetraalkylammonium ions and metal impurities through a container filled with a cation exchange resin of a hydrogen ion form or a tetraalkylammonium ion form to reduce the content of the metal impurities in the liquid to be treated, wherein the degree of cross-linking of the cation exchange resin is from 16% to 24%. The details of the method for recovering the aqueous solution of the tetraalkylammonium salt according to the present invention are the same as those described above for the purification method according to the present invention, and description thereof will be omitted.

### <Apparatus for Recovering Aqueous Solution of Tetraalkylammonium Salt>

The purification apparatus according to the present invention is an apparatus for purifying a liquid to be treated which reduces the content of metal impurities in the liquid to be treated containing tetraalkylammonium ions and metal impurities as described above, but the present invention can also be said to be an apparatus for recovering a purified aqueous solution of a tetraalkylammonium salt from the liquid to be treated by reducing the content of metal impurities in the liquid to be treated containing tetraalkylammonium ions and metal impurities. That is, the liquid to be treated purified by the purification apparatus according to the present invention is a recovered aqueous solution of a tetraalkylammonium salt. The aqueous solution of the tetraalkylammonium salt can then be contacted with, for example, an anion exchange resin or electrolyzed as described above, whereby a highly pure TAAH solution can be obtained.

An apparatus for recovering an aqueous solution of a tetraalkylammonium salt according to the present invention is an apparatus for recovering an aqueous solution of a tetraalkylammonium salt from a liquid to be treated, the apparatus including an impurity removal means for passing a liquid to be treated containing tetraalkylammonium ions and metal impurities through a container filled with a cation exchange resin of a hydrogen ion form or a tetraalkylammonium ion form to reduce the content of the metal impurities in the liquid to be treated, wherein the degree of cross-linking of the cation exchange resin is from 16% to 24%. Details of the apparatus for recovering an aqueous solution of a tetraalkylammonium salt according to the present invention are the same as those described above for the purification apparatus according to the present invention, and description thereof will be omitted.

Hereinafter, the present invention will be described in detail with reference to Examples.

### EXAMPLES

Na, Mg, K, and Ca were added as metal impurities to 1000 ml of a 10 mass % aqueous solution of tetramethylammonium chloride (TMAC), and an appropriate amount of a 25 mass % aqueous solution of tetramethylammonium hydroxide (TMAH) was further added to prepare a liquid to be treated with a pH of from 8 to 10. The amount of each metal impurity added was set to be approximately equal to the amount of the metal impurity contained in the actual photoresist developing waste liquid.

### Example 1

### (Ion Exchange Step)

This example was tested by a batch method. 10 ml of AMBERJET (registered trademark) 1060H (trade name, manufactured by Organo Corporation, degree of cross-linking: 16%) was charged into a 200 ml beaker made of PFA as an H-form strongly acidic cation exchange resin. Then, 100 ml of 2.4 mass % TMAH aqueous solution as a regeneration agent containing tetraalkylammonium ions was poured into the resin, and the mixture was stirred once in 15 minutes while rotating the beaker, and the resin was immersed for a total of 1 hour to remove the supernatant liquid to such an extent that the resin did not flow out. After this operation was performed two times, an operation of adding 100 ml of ultrapure water (UPW) and gently stirring to remove the supernatant liquid was performed three times, following which the remaining TMAH was removed by washing.

### (Impurity Removal Step)

After the ultrapure water used for washing in the ion exchange step was removed to the very limit of the resin surface, 100 ml of the liquid to be treated prepared above was poured into the resin, and the mixture was stirred once in 15 minutes while rotating the beaker, and the resin was immersed for a total of 30 minutes.

### (Measurement of Metal Concentration and pH)

The supernatant liquid after immersion was collected and then pH and metal concentration were measured. pH was measured using a portable multi-water quality meter (trade name: MM42-DP, manufactured by DKK-TOA Corporation). The metal concentration was measured using Agilent 8900 triple quadrupole ICP-MS (trade name, manufactured by Agilent Technologies). Table 1 shows the reduction ratio (%) of the concentration of each metal impurity in the liquid to be treated after purification relative to the concentration of each metal impurity in the liquid to be treated before purification and the pH value of the liquid to be treated after purification. In Table 1, the characteristic values of the cation exchange resin is the catalog values of the manufacturer.

### Example 2

The ion exchange step and the impurity removal step were performed in the same manner as in Example 1 except that AMBERLITE (registered trademark) IRN99H (trade name, manufactured by Du Pont de Nemours, Inc., degree of cross-linking: 16%) was used as an H-form strongly acidic cation exchange resin, and pH and the metal concentration were measured in the same manner as in Example 1. The results are shown in Table 1.

TABLE 1

| | | Example 1 | Example 2 |
|---|---|---|---|
| cation exchange | bland | AMBERJET | AMBERLITE |
| resin | | 1060H | IRN99H |
| | particle diameter (mm) | 0.60 to 0.70 | 0.50 to 0.55 |
| | exchange capacity (eq/L-R) | ≥2.4 | ≥2.5 |
| | degree of cross-linking (%) | 16 | 16 |
| reduction ratio of | Na | 48 | 79 |
| concentration of | Mg | 65 | 86 |
| metal impurities | K | 65 | 85 |
| (%) | Ca | 71 | 87 |

| | | |
|---|---|---|
| pH | 1 | 4 |

In Example 1 and Example 2, the same volume of the cation exchange resin having the same degree of cross-linking and the same amount of regeneration agent was used in the tests. As shown in Table 1, however, pH of the liquid to be treated after purification was 1 and strongly acidic in Example 1, and pH of the liquid to be treated after purification was 4 and weakly acidic in Example 2. This is because AMBERLITE IRN99H used in Example 2 has a smaller particle diameter and a larger surface area than AMBERJET 1060H used in Example 1. That is, it is considered that in the ion exchange step, the former is more easily converted into a TMA-form, and as a consequence of the decrease in the remaining H-form resin, pH variation at the early stage of the liquid flow due to the outflow of the hydrogen ions is suppressed in the impurity removal step. In addition, it was found that the removal performance of the metal impurities was higher in Example 2 using a resin having a smaller particle diameter than in Example 1.

### Example 3

This example was tested by a column method (see FIG. 1). As an H-form strongly acidic cation exchange resin, 36 ml of AMBERLITE (registered trademark) IRN99H (trade name, manufactured by Du Pont de Nemours, Inc., degree of cross-linking: 16%) was charged into an adsorption column (PFA column of 19 mm diameter and 300 mm length), and the resin was converted into a TMA-form by a 2.5 mass % TMAH aqueous solution (ion exchange step). Subsequently, 30 BV of the liquid to be treated used in Example 1 was passed through the resin converted to the TMA-form at a flow rate of passing 5 times the resin volume per hour (impurity removal step). Note that BV (Bed Volume) represents the multiple of the volume to be passed through the resin with respect to the volume of the resin. pH and the metal concentration of the resulting effluent were measured in the same manner as in Example 1. The results are shown in Table 2.

### Example 4

This example was tested by a column method (see FIG. 2). As in Example 3, AMBERLITE (registered trademark) IRN99H (trade name, manufactured by Du Pont de Nemours, Inc., degree of cross-linking: 16%) was used as an H-form strongly acidic cation exchange resin. 36 ml of the H-form resin, which has not been converted into a TMA-form, was charged into an adsorption column similar to that of Example 3, and 30 BV of the liquid to be treated used in Example 1 was passed through the resin at a flow rate of passing 5 times the resin volume per hour (impurity removal step). pH and the metal concentration of the resulting effluent were measured in the same manner as in Example 1. The results are shown in Table 2.

TABLE 2

| | | Example 3 | Example 4 |
|---|---|---|---|
| cation exchange | bland | AMBERLITE IRN99H | |
| resin | ion-form | TMA-form | H-form |
| | degree of closs-linking (%) | 16 | 16 |
| reduction ratio of | Na | >94 | 88 |
| concentration of | Mg | >94 | 93 |
| metal impurities | K | >94 | >94 |
| (%) | Ca | >94 | >94 |

| | | |
|---|---|---|
| pH | 6 | 3 |

As shown in Table 2, in the impurity removal step, the content of the metal impurities was able to be greatly reduced in both Example 3 in which the cation exchange resin of the TMA-form was used as the cation exchange resin and Example 4 in which the cation exchange resin of the H-form was used. In particular, in Example 3, where the liquid to be treated was passed through the resin after it was converted to the TMA-form in advance in the ion exchange step, the pH variation was small because the metal impurities and TMA were ion-exchanged in the impurity removal step. In addition, Na removing performance of Example 3 was also better than that of Example 4.

When the results of Examples 1 and 2 are compared with the results of Examples 3 and 4, the removal performance of the metal impurities is higher and the pH variation is also smaller in the latter, which is generally due to the higher purification efficiency of the column method than the batch method.

### Examples 5 to 6 and Comparative Examples 1 to 2

### (Measurement of Perfect Sphericity)

5 ml of each of the H-form cation exchange resin shown in Table 3 were poured into a 200 ml beaker made of PFA. Then, 50 ml of 25 mass % TMAH aqueous solution was poured into the resin as a regeneration agent containing tetraalkylammonium ions, mixed, and immersed for 2 hours. The supernatant liquid was then removed and the resin in the beaker was washed three times with ultrapure water (150 ml in total). This step corresponds to the ion exchange step of the present invention and was performed to confirm the presence or absence of cracking of the resin under the condition that the concentration of TMAH is higher than usual. The perfect sphericity of the resulting resin was measured by the following method.

500 resins were observed using a microscope (trade name: Digital Microscope, manufactured by KEYENCE Corporation), and the ratio (perfect spherical ratio) of the perfect spherical solid to the observed total solid was determined according to the following equation:

Perfect Sphericity (%)=((500−Number of solids with cracks or chipping)/500)×100.

The results are shown in Table 3 together with the degree of cross-linking. In Table 3, AMBERLYST (registered trademark) 16WET (trade name) used in Comparative Example 1 and AMBERLITE (registered trademark) IRN97H (trade name) used in Comparative Example 2 are both manufactured by Du Pont de Nemours, Inc.

TABLE 3

| | | degree of | perfect |
|---|---|---|---|
| | cation exchange resin | cross-linking (%) | sphericity (%) |
| Example 5 | AMBERJET 1060H | 16 | 100 |
| Example 6 | AMBERLITE IRN99H | 16 | 100 |
| Comparative Example 1 | AMBERLYST 16wet | 12 | 98 |
| Comparative Example 2 | AMBERLITE IRN97H | 10 | 91 |

As shown in Table 3, Examples 5 and 6 using highly cross-linked strongly acidic cation exchange resins showed high perfect sphericity. In other words, these resins were found to be resistant to cracking even in aqueous TMAH solution having a higher TMA ion concentration, even after repeated use in the ion exchange step, the impurity removal step, or the like.

On the other hand, Comparative Examples 1 and 2 using a resin having a degree of cross-linking lower than the range defined in the present invention had a perfect sphericity of from 91% to 98%. These resins were found to be more likely to crack due to repeated use and the like, and the ion exchange resin matrix was more likely to be damaged than the resins used in the Examples.

### EXPLANATIONS OF LETTERS OR NUMERALS

1: Adsorption column
2: Storage tank (liquid to be treated)
3: Storage tank (TAAH)
4: Storage tank (acid)
5: Storage tank (effluent)
6: Pump
7: Ultrapure water line
8: pH meter
9: Electric conductivity meter
10: Waste liquid line
11: Adsorption column
12: Storage tank (liquid to be treated)
13: Storage tank (TAAH)
14: Storage tank (effluent)
15: Pump
16: Ultrapure water line
17: pH meter
18: Electric conductivity meter
19: Waste liquid line

1. A method for purifying a liquid to be treated, the method comprising:
passing a liquid to be treated containing tetraalkylammonium ions and metal impurities through a container filled with a cation exchange resin of a hydrogen ion form or a tetraalkylammonium ion form to reduce a content of the metal impurities in the liquid to be treated,
wherein a degree of cross-linking of the cation exchange resin is from 16% to 24%.

2. The method for purifying the liquid to be treated according to claim 1,
wherein the cation exchange resin is in the tetraalkylammonium ion form, the method further comprising:
before the impurity removal step, passing a regeneration agent containing tetraalkylammonium ions through a container filled with a cation exchange resin of a hydrogen ion form to convert the cation exchange resin of the hydrogen ion form into a cation exchange resin of the tetraalkylammonium ion form,
wherein the cation exchange resin of the tetraalkylammonium ion form obtained in the ion exchange step is used in the impurity removal step.

3. The method for purifying the liquid to be treated according to claim 1, wherein a particle diameter (harmonic average diameter) of the cation exchange resin is from 500 μm to 560 μm in the hydrogen ion form.

4. The method for purifying the liquid to be treated according to claim 1, the method further comprising: regenerating the cation exchange resin that has been in contact with the liquid.

5. The method for purifying the liquid to be treated according to claim 1, wherein the liquid to be treated is a solution derived from a waste liquid discharged in developing a photoresist.

6. An apparatus for purifying a liquid to be treated, the apparatus comprising:
an impurity removal means for passing a liquid to be treated containing tetraalkylammonium ions and metal impurities through a container filled with a cation exchange resin of a hydrogen ion form or a tetraalkylammonium ion form to reduce a content of the metal impurities in the liquid to be treated,
wherein a degree of cross-linking of the cation exchange resin is from 16% to 24%.

7. The apparatus for purifying the liquid to be treated according to claim 6,
wherein the cation exchange resin is in the tetraalkylammonium ion form, the apparatus further comprising:
an ion exchange means for passing a regeneration agent containing tetraalkylammonium ions through a container filled with a cation exchange resin of a hydrogen ion form to convert the cation exchange resin of the hydrogen ion form into a cation exchange resin of the tetraalkylammonium ion form,
wherein the cation exchange resin of the tetraalkylammonium ion form obtained by the ion exchange means is used for the impurity removal means.

8. The apparatus for purifying the liquid to be treated according to claim 6, wherein a particle diameter (harmonic average diameter) of the cation exchange resin is from 500 μm to 560 μm in the hydrogen ion form.

9. (canceled)

10. An apparatus for recovering an aqueous solution of a tetraalkylammonium salt from a liquid to be treated, the apparatus comprising:
an impurity removal means for passing a liquid to be treated containing tetraalkylammonium ions and metal impurities through a container filled with a cation exchange resin of a hydrogen ion form or a tetraalkylammonium ion form to reduce a content of the metal impurities in the liquid to be treated,
wherein a degree of cross-linking of the cation exchange resin is from 16% to 24%.
